SEQUENCE LISTING (4 sequences)

SEQ ID NO: 1 - protein, 8 aa, Sus barbatus
LMYPTYLK

SEQ ID NO: 2 - protein, 8 aa, Artificial Sequence
  Description of Artificial Sequence This peptide was synthetically generated.
XQWAVXHL

SEQ ID NO: 3 - protein, 11 aa, Artificial Sequence
  Description of Artificial Sequence This peptide was synthetically generated.
XPKPXQXFXLX

SEQ ID NO: 4 - protein, 8 aa, Artificial Sequence
  Description of Artificial Sequence This peptide was synthetically generated.
XCYXXXXT

(19) United States
(12) Patent Application Publication
(10) Pub. No.: US 2003/0050233 A1
(43) Pub. Date: Mar. 13, 2003

(54) Peptide combination for the treatment of cancer

(75) Inventors: Anand C. Burman, Ghaziabad (IN); Rama Mukherjee, Ghaziabad (IN); Sudhanand Prasad, Ghaziabad (IN); Manu Jaggi, Ghaziabad (IN); Anu T. Singh, Ghaziabad (IN)

Correspondence Address: Ladas & Perry, 26 West 61 Street, New York, NY, 10023, US

(73) Assignee: DABUR ONCOLOGY PLC

(21) Appl. No.: 09/896,903

(22) Filed: Jun. 29, 2001

(51) Int. Cl. A61K 38/17

(52) U.S. Cl. 514/12

(57) ABSTRACT

The present invention relates to a combination of peptides that may be used for treatment of cancer. The combination of peptides modulates multiple cellular pathways implicated in cell proliferation by altering the levels of key intracellular molecules thereby showing a broad spectrum of anticancer activity. The invention also relates to a pharmaceutical composition containing a combination of such peptide analogs.

### FIELD OF INVENTION

The present invention relates to a combination of peptides that may be used for treatment of cancer. The combination of peptides modulates multiple cellular pathways implicated in cell proliferation by altering the levels of key intracellular molecules thereby showing a broad spectrum of anticancer activity. The invention also relates to a pharmaceutical composition containing a combination of such peptide analogs.

### BACKGROUND

The anticancer drugs currently used for the treatment of adenocarcinomas have a limited spectrum of antitumor activity and a narrow therapeutic index. For the most part, the current choices for the oncologist are among alkylating agents, antimetabolites, DNA binders, tubulin interactive antimitotics, topoisomerase inhibitors, anti-hormones, and a few other agents of mixed or unknown mechanisms. Most of these drugs as a group are similar, not only with respect to the spectrum of clinical antitumor activity and toxicity, but also with respect to their mechanism of action.

The increasing understanding of cellular and molecular biology of normal cell growth and proliferation appears to offer potentially important new targets for drug design and synthesis. A quantum leap in effective cancer chemotherapy involves rational drug design strategies based on new biochemical and molecular targets. In particular, there is considerable current interest in intracellular signaling pathways, which mediate the effects of peptide growth factors. There has been an increasing interest in the use of peptide hormone antagonists or analogues as potential therapeutic agents, which act through highly specific receptors that are significantly overexpressed in cancer cells.

We have previously shown in our US patent (U.S. Pat. No. 6,156,725; Mukherjee et al) and Australian patent (707,158; Mukherjee et al) that vasoactive intestinal peptide (VIP), somatostatin, substance P and bombesin are secreted by some human tumor cells and that there are specific high affinity binding sites for these peptides on these cells. The four peptides were also shown to bind to tumor cells. The antagonist/analogs of these peptides were shown to have anti-proliferative activity on certain cancer cells, more specifically adenocarcinomas. A combination of the peptide antagonists/analogs was also shown to cause tumor regression in a nude mice xenograft model. It was hypothesized by us that there exists an autocrine mechanism for cell proliferation where the peptides are secreted by tumor cells and transduce cellular signals through specific cell surface receptors leading to cell proliferation. The analogs/antagonists to these peptides may then abrogate/block these cellular signals linked to proliferation. Further the antiangiogenic potential of the antagonist/analogs has also been described by us previously (Mukherjee et al; U.S. application Ser. No. 09/248,381 and PCT application WO 00/047221).

We have also described in our U.S. patents applications (Ser. Nos. 09/630,333; 09/630,345; 09/629,642 & 09/629,371, Burman et al) novel antiproliferative analogs of VIP, somatostatin, bombesin and substance P that are useful in the treatment of cancer. These analogs incorporate α,α-dialkylated amino acids and show antiproliferative activity in a number of human tumor cell lines. Further, they caused partial tumor regression in nude mice xenografts when administered in a dose of approximately 4-25 μg/mouse. However, when used individually, these peptides had a narrow spectrum of activity with moderate levels of cytotoxicity. We have previously shown the synergistic in vivo tumor efficacy of these peptides in our EPO application No. 96309012.1 and we propose the use of a combination of these peptides for the treatment of cancer.

Some of the neuropeptide analogs have previously been reported to cause apotosis in cancer cells. The broad-spectrum antagonist of neuropeptide receptor, [D-Arg1, D-Phe5, D-Trp7,9, Leu11]substance P, induced apoptosis selectively in human small cell lung carcinoma (SCLC) cells, which express gastrin-releasing peptide receptor, but not in other types of tumor cells as well as normal cells. (Anticancer Res 2000 September-October; 20(5A):3123-9). It has also been shown that somatostatin, acting via the Src homology 2 bearing tyrosine phosphatase SHP-1, exerts cytotoxic action in MCF-7 cells, and triggers cell acidification and apoptosis. (J Biol Chem Mar 31, 2000;275(13):9244-50).

Further, the antiangiogenic effect of some peptide analogs has also been previously demonstrated. A VIP receptor antagonist, VIPhybrid, inhibited the increase in cAMP and vascular endothelial growth factor (VEGF) mRNA caused by VIP. By ELISA, VEGF was detected in the conditioned media exposed to the lung cancer cell lines. These results suggest that VEGF synthesis in and secretion from lung cancer cells can be regulated by agents, which cause adenylyl cyclase activation (Lung Cancer 2001 March; 31(2-3):203-212 ). There is further evidence that suggests that there is an inhibitory effect of octreotide on RPE cell proliferation of bovine RPE cells and on the increased proliferation of bovine RPE cells induced by platelet derived growth factor (PDGF) and basic fibroblast growth factor (bFGF). An enhanced inhibitory effect was found for the combination of octreotide and VEGF (Ophthalmologe 2000 November; 97 (11):737-41). In a separate study, the effect of somatostatin-14 (SRIF) and ocreotide (sandostatin) on proliferation activity and VEGF release from cultured murine endothelial cells HECa10 was studied in vitro. Somatostatin releasing inhibitory factor (SRIF) diminished the proliferative activity of cultured cells vs. controls. Although the antiproliferative effect of SRIF and ocreotide on mouse endothelial cells was shown, the inhibitory effect of tested peptides on VEGF secretion in vitro was not demonstrated (Biochem Biophys Res Commun 2000 Feb 16;268(2):567-71).

The adenocarcinomas express and secrete multiple growth factors viz. platelet derived growth factor (PDGF), epidermal growth factor (EGF) and transforming growth factor (TGF) alpha. The binding of growth factors to their respective receptors activates a cascade of intracellular pathways, specifically phosphorylation events mediated by protein kinases and phosphatases, which modulate the activity of a variety of cellular transcription factors. Aberrations in these signal-induced events are associated with cancer development and/or progression of cancer.

The ability to repress intracellular signal-induced response pathways is an important mechanism in negative control of gene expression. Selective disruption of such pathways allows the development of therapeutic agents capable of treating a variety of disease states related to improper activation and/or expression of specific transcription factors.

The cellular signaling mediated by receptors coupled to G proteins, as those for regulatory peptides are transduced through the cAMP-adenylyl cyclase system. The mitogenic response of the cells to growth factors and regulatory peptides is influenced by intracellular concentrations of cAMP, which in turn activates the cAMP dependent protein kinases (PKA). cAMP cooperates with a variety of hormones and growth factors to synergistically stimulate the proliferation of different type of eukaryotic cells. We have investigated the effects of the peptide combination on cAMP levels.

The secretion of multiple growth factors viz. PDGF, EGF and TGF (alpha) by the adenocarcinomas led us to investigate the effect of the peptide combination on growth factor mediated signaling. The receptor tyrosine kinases (RTK) are transactivated by G protein coupled receptors (GPCR). Platelet derived growth factor (PDGF), epidermal growth factor (EGF) and insulin like growth factor 1 (IGF 1) are tyrosine phosphorylated subsequent to GPCR activation. The phosphorylated growth factors in turn recruit multiple accessory proteins to activate the mitogen activated protein kinases (MAPK). Human adenocarcinomas have increased constitutive MAPK activity, and the blockade of this protein kinase suppresses tumour growth in vitro and in vivo. We have investigated the effect of peptide combination on growth factor mediated proliferation and its effects on mitogen activated protein kinase (MAPK).

Membrane associated tyrosine phosphatases dephosphorylate specific targets, thus functionally opposing the action of tyrosine kinases. Inhibition of tyrosine phosphatase reversibly induces transformation of cultured cells in a dose dependent manner in vitro. Induction of tyrosine phosphatases in cancer cells leads to cell differentiation and reversal of transformed phenotype.

### SUMMARY OF INVENTION

In our present application, we have used a combination of peptide analogs, which as compared to individual peptides, causes a significantly higher cytotoxicity in tumor cells. This led us to investigate the pathways and the key signaling molecules that were altered by the peptide combination.

We investigated whether multiple cellular pathways and targets are modulated by the peptide combination in neoplastic cells. The growth factor dependent proliferation, the cAMP-adenylate cyclase, the mitogen activated protein kinase (MAPK) and the tyrosine phosphatases, were modulated by the peptide combination of this invention. Thus the peptide antagonists in the combination, compete with the receptors for the native peptides at the plasma membrane and indirectly regulate their intracellular tyrosine protein kinase activity and its key intermediates and the mitogenic signaling complexes regulated by the peptides. The peptide combination restored wild type p53 expression and downregulated the levels of the antiapoptotic protein bc12 thus triggering the apoptotic cell death. Further, it activates active Caspase-3, a key enzyme in the apoptotic pathway. Additionally the combination is also antiangiogenic as it inhibits “tube-like structure” formation and migration of endothelial cells concomitantly with a decrease in the levels of VEGF.

Thus, the combination of peptides blocks several of the pathways implicated in cell proliferation by altering the levels of key molecules involved and is therefore able to cause not only significantly higher cell death but also displays a broader spectrum of anticancer activity. The peptide combination causes significant reduction in cAMP levels in adenocarcinomas of the stomach and breast. It inhibits epidermal growth factor (EGF) dependent proliferation in the adenocarcinomas of the pancreas and ovary. The peptide combination causes the induction of active Caspase-3 in ovarian cancer cells, and it causes decrease in levels of VEGF in breast cancer. Therefore, having conducted several experiments, we have optimized a combination of peptides with high cytotoxic and broad spectrum activity. The anticancer effect of the present invention is mediated by altering the multiple cellular pathways in the neoplastic cells thereby leading to apoptosis and antiangiogenic activity.

The present invention relates to a composition useful for killing or inhibiting the growth and/or multiplication of tumor and/or cancer cells. The composition may suitably comprise, consist of, or consist essentially of a therapeutically effective combination of a peptide analog of somatostatin, a peptide analog of a vasoactive intestinal peptide, a peptide analog of bombesin and a peptide analog of substance P. In a preferred embodiment, a pharmaceutically effective carrier, diluent, or solvent is used with the peptide analogs. The invention also provides a method of treatment for humans, mammals or other animals suffering from cancer and cancer associated angiogenesis and metastasis. The method may suitably comprise, consist of, or consist essentially of administering a therapeutically effective dose of the composition so as to inhibit tumor or cancer associated angiogenesis or metastasis and to kill, inhibit the growth or inhibit the multiplication of cancer or tumor cells by inducing apoptosis.

Another aspect of the invention provides a method for treating a mammal (including a human being) afflicted with cancer.

### BRIEF DESCRIPTION OF THE FIGURE

FIG. 1 shows in vivo antitumor activity on subcutaneous administration of the peptide combination on PTC xenografts.

### DETAILED DESCRIPTION OF THE INVENTION

This invention comprises, consists of, or consists essentially of compositions of combinations of at least 2 peptides selected from peptides of SEQ ID NO: 1, SEQ ID NO: 2, SEQ ID NO: 3 and SEQ ID NO: 4. The combinations may be selected from:

1) SEQ ID NO: 1 and SEQ ID NO: 2

2) SEQ ID NO: 1 and SEQ ID NO: 3

3) SEQ ID NO: 1 and SEQ ID NO: 4

4) SEQ ID NO: 2 and SEQ ID NO: 3

5) SEQ ID NO: 2 and SEQ ID NO: 4

6) SEQ ID NO: 3 and SEQ ID NO: 4

7) SEQ ID NO: 1, SEQ ID NO: 2 and SEQ ID NO: 3

8) SEQ ID NO: 2, SEQ ID NO: 3 and SEQ ID NO: 4

9) SEQ ID NO: 1, SEQ ID NO: 3 and SEQ ID NO: 4

10) SEQ ID NO: 1, SEQ ID NO: 2 and SEQ ID NO: 3

11) SEQ ID NO: 1, SEQ ID NO: 2, SEQ ID NO: 3 and SEQ ID NO: 4.

The amino acid sequences represented by SEQ ID NO: 1, SEQ ID NO: 2, SEQ ID NO: 3 and SEQ ID NO: 4. are described below. A pharmaceutically acceptable salt of SEQ ID: 1, SEQ ID NO: 2, SEQ ID NO: 3 or SEQ ID NO: 4 can be used in place of the respective SEQ ID: 1, SEQ ID NO: 2, SEQ ID NO: 3 or SEQ ID NO: 4. The concentration of each peptide in each combination is 10⁻⁶ to 10⁻¹⁰ m, preferably 10⁻⁸ to 10⁻⁹ m.

The methods of this invention comprise, consist essentially or consist of administering to a human or animal, preferably a mammal, a therapeutically effective combination of this invention. The combinations of this invention can be used to kill or inhibit the multiplication of tumor cells or cancer cells. The combinations are especially effective in treating breast, ovarian, colon, lung, pancreatic, prostate, stomach and oral cancer and in treating skin fibroblasts. The combinations of this invention can be used to induce caspase enzyme, downregulate intracellular levels of cAMP; inhibit secretion of vascular endothelial growth factor (VEGF); down regulate intracellular levels of mitogen activated protein kinase (MAPK); upregulate intracellular levels of tyrosine phosphasphatase; and down regulate epidermal growth factor dependent proliferation.

The ratio of peptides in the formulations can vary such that the weight of one peptide may be between 1 to 3 times the weight of another peptide. The total weight of a single dose is between 0.01 to 50.0 mg. This conforms to a dose of 0.05-500 μg/Kg. body weight of the human or animal. These peptides have their best in vitro or systemic biological activity (anticancer) when their individual concentrations range between 10⁻⁶ M to 10⁻¹⁰ M (preferably 10⁻⁸ M to 10⁻⁹ M). It is not necessary that the weight of all the peptides in the dose should be the same. The weight of the peptide in a dose would depend upon many factors such as (i) bioavailability of peptide, (ii) half-life of peptide, and (iii) number and affinity of receptors for a particular peptide on tumor.

An effective dose of the combination ranges from 0.05-500 μg/Kg. body weight of the mammal, with the dose dependent on inter alia the effects sought, the manner of administration, the peptide selected, and the cancer being treated. Systemic administration refers to oral, rectal, nasal, transdermal, and parentral (i.e., intramuscular, intravenous, and subcutaneous). In accordance with good clinical practice, it is preferred to administer the composition at a dose that will produce anticancer effects without causing undue harmful side effects. The composition may be administered either alone or as a mixture with other therapeutic agents.

The composition may optionally and preferably contain pharmaceutically acceptable diluents, excipients, solvents, binders, stabilizers and the like. Such diluents may include buffered saline, isotonic NaCl, Ringer's solution, water, distilled water, polyethylene glycol (neat or in water), Tween in water, dimethylsulfoxide upto 50% in water, propylene glycol (neat or in water), phosphate buffered saline, balanced salt solution, glycerol, and other conventional fluids that are suitable for parentral administration.

Pharmaceutical compositions which provide 0.01 to 20.0 mg of the combination per unit dose are preferred and are conventionally prepared as tablets, lozenges, capsules, powders, aqueous or oily suspensions, syrups, elixirs, and aqueous solutions. The nature of the pharmaceutical composition employed will, of course, depend on the desired route of administration.

The present invention is further described in detail with reference to the following examples, which are given for the purpose of merely illustrating the invention without limiting it.

### EXAMPLE 1

### Synthesis of Peptides

The peptides in the present invention have been generated by solid phase technique following the Fmoc strategy, on a semi automatic peptide synthesizer (CS Bio, Model 536), using optimum side chain protection. The peptides were assembled from C-terminus to N-terminus. Peptides amidated at the carboxy-terminus were synthesized using the Rink Amide resin and free carboxy-terminus peptide using Wang Resin.

After the assembly of the peptide was completed, the amino-terminal Fmoc group was removed and then the peptide-resin was washed with methanol and dried. The peptides were then deprotected and cleaved from the resin support by treatment with trifluoroacetic acid, crystalline phenol, ethanedithiol, thioanisole and de-ionized water for 1.5 to 5 hours at room temperature. The crude peptide was obtained by precipitation with cold dry ether. It was further dissolved in water and lyophilized.

The resulting crude peptide was purified by preparative high performance liquid chromatography using a C-18 reverse phase column on a Preparative HPLC system using a gradient of 0.1% TFA in acetonitrile and water. The eluted fractions were reanalyzed on analytical HPLC system. Acetonitrile was evaporated and the fractions were lyophilized to obtain the pure peptide. The identity of each peptide was confirmed by mass spectroscopy. The peptides yielded as trifluoracetate salt. The peptides may contain other pharmaceutically acceptable salts. Salts encompassed within the term “pharmaceutically acceptable salt” refer to nontoxic salts of the compounds of this invention. Representative salts and esters include the following: acetate, ascorbate, benzoate, citrate, oxalate, stearate, trifluoroacetate, succinates, tartarate, lactate, fumarate, gluconate, glutamate, phosphate/diphosphate, valerate and the like.

The following abbreviations are used for uncommon amino acids:

Orn=Ornithine

Pen=Penicillamine

Aib=α-Aminoisobutyric acid

Ac5c=1-Aminocyclopentane carboxylic acid

The sequence of the VIP receptor binding inhibitor is:

Leu-Met-Tyr-Pro-Thr-Tyr-Leu-Lys-OH (SEQ ID NO:1) which is described in our U.S. patent application Ser. No. 09/630,345

The sequence of Bombesin antagonist is:

Butanoyl-D-Phe-Gln-Trp-Ala-Val-Aib-His-Leu-NH₂ (SEQ ID NO: 2) which is described in our U.S. patent application No. 09/630,333.

The sequence of Substance P antagonist is:

D-Arg-Pro-Lys-Pro-D-Phe-Gln-D-Trp-Phe-D-Trp-Leu-Ac5c-NH₂ (SEQ ID NO:3) which is described in our U.S. patent application No. 09/629,642.

The sequence of Somatostatin analogue is:

D-Phe-Cys-Tyr-D-Trp-Om-Ac5c -Pen-Thr-NH₂ (SEQ ID NO:4)

(There is a disulphide bond between Cys and Pen amino acids) which is described in our U.S. patent application No. 09/629,371.

### EXAMPLE 2

### The Combination was Prepared in the Following Way

A stock solution for each of the four peptides (SEQ ID NO:1, SEQ ID NO: 2, SEQ ID NO:3 and SEQ ID NO: 4) is prepared with a pH of approximately 3.5 to 7.0 but preferably 4.0 to 5.5. Although sterile phosphate buffered saline was used to prepare each stock solution for the testing described in the following example, other diluents may be used such as buffered saline, isotonic NaCl, Ringer's solution, water, distilled water, polyethylene glycol (neat or in water), Tween in water, dimethylsulfoxide upto 50% in water, propylene glycol (neat or in water), phosphate buffered saline, balanced salt solution, glycerol, and other conventional fluids that are suitable for parentral administration. To obtain a pH in the range of approximately 3.5 to 7.0, for each stock solution, the pH can be adjusted by using 1N HCl for lowering the pH or 1N NaOH for raising the pH, although other buffers such as citrate buffer, phosphate buffer and the like or other conventional agents for adjusting the pH can be used. The stock solution of each peptide is then mixed in appropriate dilutions so as to give a final concentration of 10⁻⁷ to 10⁻¹¹ M but more preferably in the range of 10⁻⁸ to 10⁻¹⁰ M. A stock solution for the pharmaceutically acceptable salts is prepared in the same way.

### Formulation of a Dose of the Combination for Parentral Administration

A dose of the formulation of combination was prepared in the following way. A stock solution of each of the four peptides SEQ ID NO:1, SEQ ID NO:2, SEQ ID NO:3 and SEQ ID NO:4 was first prepared using sterile phosphate buffered saline with an approximate pH of 4.0 to 5.5. Aliquots of the stock solutions of the peptides were mixed together to prepare formulations containing combinations of two peptides, formulations containing three peptides or formulations containing four peptides. The ratio of peptides in the formulations can vary such that the weight of one peptide may be between 1 to 3 times the weight of another peptide. The total weight of a single dose is between 0.01 to 50.0 mg. This conforms to a dose of 0.05-500 μg/Kg. body weight of the mammal. These peptides have their best in vitro or systemic biological activity (anticancer) when their individual concentrations range between 10⁻⁶ M to 10⁻¹⁰ M (preferably 10⁻⁸ M to 10⁻⁹ M). To achieve therapeutically effective systemic concentrations, a dose of 0.05-500 μg/Kg.B.Wt of the mammal needs to be administered. Further, it is not necessary that the weight of all the peptides in the dose should be the same. The weight of the peptide in a dose would depend upon many factors such as (i) bioavailability of peptide, (ii) half-life of peptide, and (iii) number and affinity of receptors for a particular peptide on tumor. The combinations may be as follows:

TABLE I

| S. | |
|---|---|
| Number | Combination |
| 1. | SEQ ID NO:1 + SEQ ID NO:2 |
| 2. | SEQ ID NO:1 + SEQ ID NO:3 |
| 3. | SEQ ID NO:1 + SEQ ID NO:4 |
| 4. | SEQ ID NO:2 + SEQ ID NO:3 |
| 5. | SEQ ID NO:2 + SEQ ID NO:4 |
| 6. | SEQ ID NO:3 + SEQ ID NO:4 |
| 7. | SEQ ID NO:1 + SEQ ID NO:2 + SEQ ID NO:3 |
| 8. | SEQ ID NO:1 + SEQ ID NO:2 + SEQ ID NO:4 |
| 9. | SEQ ID NO:1 + SEQ ID NO:3 + SEQ ID NO:4 |
| 10. | SEQ ID NO:2 + SEQ ID NO:3 + SEQ ID NO:4 |
| 11. | SEQ ID NO:1 + SEQ ID NO:2 + SEQ ID NO:3 + SEQ ID NO:4 |

### EXAMPLE 3

### In Vitro Cytotoxicity Studies on the Combination

The combination SEQ ID NO:1+SEQ ID NO:2+SEQ ID NO:3+SEQ ID NO: 4, all in 10⁻⁸ M concentration was tested for cytotoxicity against 12 human tumor cell lines. It was also tested against primary human colon adenocarcinoma cells and other colon cancer cell lines. Briefly, a one day MTT cytotoxicity assay was performed, which is based on the principle of uptake of MTT (3-(4,5-dimethylthiazol-2-yl)-2,5-diphenyl tetrazolium bromide), a tetrazolium salt, by the metabolically active cells where it is metabolized by active mitochondria into a blue colored formazan product that is read spectrophotometrically [Mosmann, 1983]. MTT was dissolved in phosphate buffered saline with a pH of 7.4 to obtain an MTT concentration of 5 mg/ml; the resulting mixture was filtered through a 0.22 micron filter to sterilize and remove a small amount of insoluble residue. For each type of tumor cell, 20,000 to 50,000 cells were seeded in a 96-well culture plate and incubated with the combination in a CO₂ incubator for 24 hours. The final concentration of each peptide in the combination was 10⁻⁸ M. Control cells not treated with the combination were similarly incubated. The assay was terminated after 24 hours by adding 100 ug (20 ul) of MTT to each well, then incubating for additional one hour, and finally adding 50 ul of 10% SDS-0.01N HCl to each well to lyse the cells and dissolve formazan. After incubating for one hour, the plate was read spectrophotometrically at 540 nm and the cytotoxicity percentage calculated.

### Cytotoxic Effect of the Combination on Human Tumor Cell Lines

Experiments were conducted to study the cytotoxic effect of the combination on 12 human tumor cell lines using the three-day MTT cytotoxic assay. These cell lines were K562 (human leukemia), MOLT-4 (human lymphoma), L132, A549 (human lung carcinoma), MCF-7, HBL100, MDA.MB.453 (human breast), SW620, PTC, CoLo205, HT29, CaCO.2 (human colon), HuTu80 (human duodenum), Hu746T (human stomach), SKO.007 (human myeloma), SK.MEL.28 (human melanoma). Briefly, cells from the 12 human tumor cell lines were incubated in a 96-well culture plate (approximately 50,000 cancer cells in each well) for 72 hours at 37° C. in a CO₂ incubator. The combination SEQ ID NO: 1+SEQ ID NO: 2+SEQ ID NO: 3+SEQ ID NO: 4, all at 10⁻⁸ M concentration (20 ul per well) was added to the wells of all the treated samples at time 0, 24, and 48 hours. The controls were cells from 12 tumor cell lines that were not treated with the combination. At the end of 72 hours, stock MTT solution was added to each well, and incubation continued for one additional hour. After adding SDS-0.01N HCl, the plate was read at 540 nm. The percent cytotoxicity caused by the combination in each of the 12 cell lines is listed in Table-II.

TABLE II
Percentage Cytotoxicity Caused By The Combination
In Human Tumor Cell Lines

| Cell line | Tumor type | Percent Cytotoxicity |
|---|---|---|
| Colon | PTC | 92.4 ± 3.4 |
| | CoLo205 | 73.2 ± 3.1 |
| | SW620 | 64.1 ± 3.4 |
| Lung | L132 | 38.9 ± 4.9 |
| Breast | HBL100 | 54.1 ± 3.9 |
| | MCF-7 | 48.9 ± 5.2 |
| Pancreas | MiaPaCa.2 | 71.1 ± 3.2 |
| Prostate | DU145 | 37.4 ± 6.4 |
| Ovary | PA-1 | 76.9 ± 4.2 |
| Stomach | HuTu80 | 70.4 ± 4.3 |
| Oral | KB | 64.0 ± 3.1 |
| Skin fibroblast | A431 | 70.5 ± 5.6 |

### EXAMPLE 4

### Cytotoxicity Abrogation Assays for Identification of Cellular Signaling Pathways Modulated by Peptide Combination

Human colon adenocarcinoma cells cultured to 70% confluence were harvested using 0.05% trypsin-0.2 mM EDTA solution. Subsequently, the cells were re-plated in medium supplemented with 10% FCS, in 96 well micro titer tissue culture plates at a density of 5000 cells/well. The plates were incubated overnight to allow complete reattachment of the cells. The medium was replaced with RPMI-1 640 containing 2.5% FCS. The specific cellular signaling inhibitors for different signaling pathways were added in pre-determined optimal non toxic concentrations (in pM-nM range) once every 24 hours, for a period of 72 hours. The control cells were incubated with the vehicle alone. After 72 hours, the cell viability was quantitated by the MTT assay.

The inhibitors to specific cellular signaling pathways were added individually along with the peptide combination SEQ ID NO: 1+SEQ ID NO: 2+SEQ ID NO: 3+SEQ ID NO: 4, all at 10⁻⁹M concentration once every 24 hours, for a period of 72 hours. The control cells were incubated with the peptide combination or with the vehicle alone. The cell viability was quantitated by the Formazan based MTT assay. The percent abrogation in the cytotoxicity of the peptide combination when co-incubated with the specific cellular signaling inhibitor was calculated.

The treatment of cells with the peptide combination and the above inhibitors, demonstrated that specific signaling inhibitors caused a time-dependent abrogation of the anti-tumor activity of the peptide combination, as shown in the Table-III. Thus multiple cellular signaling pathways viz. the protein kinases, tyrosine kinases, tyrosine phosphatases and the cAMP dependent protein kinases are the key cellular pathways modulated by the peptide combination to bring about its anticancer effects.

TABLE III
Time Kinetics Of The Abrogation Of Cytotoxic Activity Of Peptide
Combination By Different Inhibitors

| | 48 hours | | | 72 hours | | |
|---|---|---|---|---|---|---|
| | Conc. of | | Abrogation, | Conc. of | | Abrogation, |
| Inhibitor | inhibitor | | % control | inhibitor | | % control |
| Indomethacin | — | | — | — | | — |
| Staurosporine | 80 | pM | 13% | 80 | pM | 27% |
| Tyrphostin 47 | 37 | nM | 13% | — | | — |
| Piceatannol | 400 | nM | 15% | — | | — |
| cAMP-S (Rp | 31 | μM | 27% | — | | — |
| isomer) | | | | | | |
| Bisindolylmaleimide | — | | — | — | | — |
| Okadiac acid | 31 | nM | 25% | — | | — |
| Sodium | — | | — | 8 | nM | 72% |
| orthovanadate | | | | | | |

### EXAMPLE 5

### Effect of Peptide Combinations on Camp Levels in Breast Cancer Cells

Enzyme linked sandwich immunosorbent assay was carried out on breast cancer cells (MF7) to study the modulation in levels of cAMP upon treatment with the peptides and their combinations. The cells were cultured in sterile 6 well plates at a density of 0.5×10⁶/well in DMEM supplemented with 10% FCS, and allowed to adhere overnight. The medium was changed to DMEM and the cells were treated with appropriate concentrations of the peptide and its combinations, as shown in Table-IV, for 30 minutes, such that in each case the final peptide concentration was 10⁻⁹M.

The cells were coincubated with Forskolin at a concentration of 1 uM. The cells were scraped using a cell scraper and pelleted. The cell pellet was suspended in appropriate resuspension buffer (50 mM Tris, containing EDTA, 0.2 mM phenyl methyl sulfonyl fluoride (PMSF), 1 ug/ml pepstatin and 0.5 ug/ml leupeptin adjusted to pH 7.4). The cell extracts were transferred to microcentrifuge tubes and centrifuged for 5 minutes at 10000×g. cAMP standards ranging in concentrations from 200 units/ml to 0 units/ml were prepared immediately prior to the start of the assay. Fluorescein isothiocyanate (FITC) conjugated monoclonal antibody directed to cAMP was added to microtitre wells precoated with primary monoclonal antibody against cAMP. 50 ul of sample lysates, and cAMP standards were added in duplicates in the microtitre wells containing both the monoclonals to cAMP. The wells were incubated at room temperature for 2 hours. The microtitre wells were washed thrice with appropriate wash buffer (PBS 50 mM, with Tween 0.5%) and the appropriate dilution of anti FITC horse radish peroxidase conjugate was added and incubated for 30 minutes. The microtitre wells were washed thrice with the wash buffer and subsequently with distilled water. Tetra-methylbenzidine was added to each well and incubated in dark for 30 minutes at room temperature. The absorbance was measured in each well at dual wavelength of 450/595 nm within 30 minutes of the addition of stop solution and the amount of cAMP present in the cell samples calculated.

The intracellular levels of cAMP on treatment with peptide subcombinations was downregulated by the peptide subcombinations by 30 minutes of the drug treatment. Table IV shows percentage inhibition in the levels of cAMP following treatment with subcombinations on MF 7 cells (breast cancer cells). The maximum inhibition in the intracellular levels of cAMP was seen with the four peptide combination of SEQ ID NO: 1+SEQ ID NO: 2+SEQ ID NO:3+SEQ ID NO:4.

TABLE IV

| | | Camp |
|---|---|---|
| | | (% of |
| S. No. | PEPTIDE (combinations) | Control) |
| 1 | SEQ ID NO:1 | 30% |
| 2 | SEQ ID NO:2 | 18% |
| 3 | SEQ ID NO:3 | 14% |
| 4 | SEQ ID NO:4 | 15% |
| 5 | SEQ ID NO:2 + SEQ ID NO:1 | 15% |
| 6 | SEQ ID NO:1 + SEQ ID NO:2 + SEQ ID NO:4 | 14% |
| 7 | SEQ ID NO:1 + SEQ ID NO:2 + SEQ ID NO:3 + | 8% |
| | SEQ ID NO:4 | |

### EXAMPLE 6

### Effect of Peptide Combinations on Epidermal Growth Factor Dependent Proliferation in Pancreatic Cancer Cells (MiaPaCa2)

Pancreatic cancer cells (MiaPaCa2) were cultured in 96 well culture plates at a density of 10000 cells/well in DMEM supplemented with 10% FCS, and allowed to adhere overnight. The medium was changed to DMEM and the cells were treated with recombinant Epidermal growth factor (EGF) in a concentration ranging from 5 nM to 1 uM. The cells were incubated for a cumulative period of 72 hours. EGF was added to the cells once every 24 hours. The control cells were not treated with EGF. The survival fraction of the cells treated with EGF was compared to that in the untreated cells by the MTT assay.

The assay was terminated by adding 100 ug (20 ul) of MTT to each well, then incubating for additional one hour, and finally adding 50 ul of 10% SDS-0.01N HCl to each well to lyse the cells and dissolve formazan. After incubating for one hour, the plate was read spectrophotometrically at 540 nm and the cell survival fraction calculated. The optimal concentration of EGF causing proliferation of the pancreatic cells was obtained. The pancreatic cancer cells were treated with the optimal concentration of EGF (5 nM) and the peptide subcombinations shown in Table V, for 72 hours, such that in each case the final peptide concentration was 10⁻⁹M.

The peptide combinations and EGF were added every 24 hours. The fraction of surviving cells was calculated for each peptide combination by the MTT assay described above. Table V below shows the survival fraction of the cells treated with EGF and different peptide combinations.

The Epidermal growth factor dependent cellular proliferation was abrogated by the peptide combinations to varying extents in vitro. This inhibition was specific for EGF as the experiments were carried out in medium free of any other growth factors. Table V shows the survival fraction of cells treated with varying peptide combinations expressed as a percent of control untreated cells. The maximal inhibition of the proliferation was blocked by four peptide combination of SEQ ID NO: 1+SEQ ID NO: 2+SEQ ID NO:3+SEQ ID NO:4.

TABLE V

| | | Survival |
|---|---|---|
| | | fraction |
| S. No. | PEPTIDES COMBINATIONS | of cells |
| 1 | SEQ ID NO:1 | 85% |
| 2 | SEQ ID NO:2 | 90% |
| 3 | SEQ ID NO:3 | 90% |
| 4 | SEQ ID NO:4 | 70% |
| 5 | SEQ ID NO:2 + SEQ ID NO:1 | 65% |
| 6 | SEQ ID NO:1 + SEQ ID NO:2 + SEQ ID NO:4 | 62% |
| 7 | SEQ ID NO:1 + SEQ ID NO:2 + SEQ ID NO:3 + | 20% |
| | SEQ ID NO:4 | |

### EXAMPLE 7

### Effect of Peptide Combinations on Expression of Tyrosine Phosphatase in Pancreatic Cancer Cells

The quantitation of tyrosine phosphatase was carried out colorimetrically by using their synthetic biotin labeled tyrosine phosphorylated peptide substrates. The enzyme reaction was quenched by the addition of a specific inhibitor, and the phosphorylated and dephosphorylated substrate is immobilized by binding to the streptavidin coated microtitre plate. The fraction of the unmetabolized substrate is determined by its anti phospho tyrosine antibody directly conjugated to peroxidase.

The cells were cultured in sterile 6 well plates at a density of 0.5×10⁶/ well in DMEM supplemented with 10% FCS, and allowed to adhere overnight. The medium was changed to DMEM. The cells were treated with appropriate concentrations peptide and their combinations shown in Table VI, for 30 minutes, such that in each case the final peptide concentration was 10⁻⁹M.

The cells were lysed in appropriate lysis buffer (50 mM Tris, containing 0.2 mM PMSF, 1 ug/ml pepstatin and 0.5 ug/ml leupeptin adjusted to pH 7.4). The cell extracts were transferred to microcentrifuge tubes and centrifuged for 5 minutes at 10000×g.

The quantitation of phosphatase enzyme activity was performed with 30 ul of specific substrates and the reaction was quenched with the addition of 100 uM sodium orthvanadate. The reaction mixture was added to streptavidin coated microtitre plate. The wells were washed thoroughly and 75 ul of Anti-Phospho Tyrosine antibody was added to the plate. After 3 washings, 100 ul of the substrate POD was added to the wells, and the absorbance was read at 405 nM.

The peptide combination induced tyrosine phosphatase activity in within 30 minutes in pancreatic cancer cells. Table-VI shows show percentage induction of typrosine phosphatase activity following treatment with the subcombinations on pancreatic cancer cell line (MiaPaCA2). The maximum induction of tyrosine phosphatase 150 % in pancreatic cancer cells (Mia PaCa2) occurred in the four peptide combination of SEQ ID SEQ ID NO:2, SEQ ID NO:3 and SEQ ID NO:4.

TABLE VI
Effect Of Peptide Combinations On Induction Of Tyrosine Phosphatase
In Pancreatic Cancer Cells (Miapaca2)

| | | % Induction |
|---|---|---|
| | | of Tyrosine |
| S. No. | Peptide Combinations | phosphatase |
| 1 | SEQ ID NO:4 | 10% |
| 2 | SEQ ID NO:3 | 12% |
| 3 | SEQ ID NO:2 + SEQ ID NO:4 | 14% |
| 4 | SEQ ID NO:2 + SEQ ID NO:4 + SEQ ID NO:3 | 40% |
| 5 | SEQ ID NO:1 + SEQ ID NO:2 + SEQ ID NO:3 + | 50% |
| | SEQ ID NO:4 | |

### EXAMPLE 8

### Effect of Peptide Combination on Caspase-3 Induction

PA-1 (Ovary) cells were cultured in 25 cm² tissue culture flasks at 37° C. in a humidified incubator containing 5% CO₂. When cultures reached pre-confluence individual peptides of the combination and different combinations of the peptides as shown in Table VII were added to different flasks such that the final concentration of each peptide in the flask was 10⁻⁸ M. The untreated flasks served as controls. The cells were incubated with drug for 6 hours.

After 6 hours of incubation cells were collected by trypsin treatment followed by centrifugation at 2000 r.p.m. for 10 minutes. The supernatant was gently removed and discarded and Lysis Buffer was added to the cell pellet (25 ul of lysis buffer per 1×10⁴ cells I a 15 ml conical tube) and incubated on ice for 10 minutes and centrifuged at 10,000-×g for 1 minute. 50 ul of supernatant(cell lysate) was transferred to the wells of a 96-well plate. 2×Reaction buffer was prepared by adding 10 ul of fresh DTT stock per 1 ml of 2×Reaction Buffer. 50 ul 2×Reaction Buffer was added to each well followed by addition of 5 ul of Caspase-3 colorimetric substrate (DEVD-pNA). The plate was incubated for 1-2 hours at 37° C. Following incubation the plate was read on a microplate reader at 405 nm-wavelength light the level of caspase enzymatic activity in the cell lysate was directly proportional to the color reaction. The induction of caspase activity by the subcombinations was calculated as a percentage of control using the formula [(Sample OD/Control OD)−1]*100.

Caspase-3 activation on treatment with peptides was seen as early as 6 hours in PA-1 (ovary) cells. Table VII shows percentage induction of caspase activity following treatment with subcombinations on PA-1 (Ovary) cell line. The maximum caspase induction of 62.4% (PA-1) and 46.4% (MDA MB 453) was seen in the four peptide combination of SEQ ID NO:1, SEQ ID NO:2, SEQ ID NO:3 and SEQ ID NO:4.

TABLE VII
Effect Of Peptide Combination On Caspase-3 Induction

| S. No. | PEPTIDE COMBINATIONS | % INDUCTION |
|---|---|---|
| 1 | SEQ ID NO:4 | 12.4 |
| 2 | SEQ ID NO:3 | 19.5 |
| 3 | SEQ ID NO:2 + SEQ ID NO:4 | 22.7 |
| 4 | SEQ ID NO:2 + SEQ ID NO:4 + | 32.9 |
| | SEQ ID NO:3 | |
| 5 | SEQ ID NO:1 + SEQ ID NO:2 + | 62.4 |
| | SEQ ID NO:3 + SEQ ID NO:4 | |

### EXAMPLE 9

### Effect of Peptide Combination on Vascular Endothelial Growth Factor Activity

Human breast adenocarcinoma cells (MDA.MB.453) were plated at the density of 8-10×10⁵ cells per 2 ml in a six well plate. After an overnight incubation of cells at 37° C., individual peptides and different combinations of the peptides as shown in Table VIII were added to different wells such that the final concentration of each peptide was 10⁻⁸ M. The untreated wells served as controls. The plates were incubated for 4 hours at 37° C. The medium was collected from all the wells (control and treated) and spun down at 2000 r.p.m to remove the cellular material. The supernatant was collected and used for ELISA (Quantikine human VEGF, R&D Systems). The assay employs the quantitative sandwich enzyme immunoassay technique. A monoclonal antibody specific for VEGF has been pre-coated onto a microplate. Standards and samples were pipeted into the wells and VEGF present was bound by the immobilized antibody. After washing away the unbound substances, an enzyme-linked polyclonal antibody specific for VEGF was added to the wells. Following a wash to remove any unbound antibody-enzyme reagent, a substrate solution was added to the wells and color developed in proportion to the amount of VEGF bound in the initial step. The optical density of each well was determined within 30 minutes using a microplate reader set to 450 nm and reference wavelength at 540 nm.

The cells showed high levels of secretion of VEGF, which was inhibited by the addition of peptide analogs. The results of the assay are summarized in the folowing Table

TABLE VIII
Effect Of Peptide Combination On Vascular
Endothelial Growth Factor Activity

| S. NO. | PEPTIDE COMBINATIONS | % INHIBITION |
|---|---|---|
| 1 | SEQ ID NO:4 | 12.4 ± 2.3 |
| 2 | SEQ ID NO:3 | 17.5 ± 1.9 |
| 3 | SEQ ID NO:2 + SEQ ID NO:4 | 19.7 ± 2.0 |
| 4 | SEQ ID NO:2 + SEQ ID NO:4 + | 24.6 ± 3.1 |
| | SEQ ID NO:3 | |
| 5 | SEQ ID NO:1 + SEQ ID NO:2 + | 29.5 ± 3.5 |
| | SEQ ID NO:3 + SEQ ID NO:4 | |

### EXAMPLE 10

As shown previously in our U.S. Applications (Ser. Nos. 09/630,333; 09/630,345; 09/629,642 and 09/629,371, Burman et al), the individual peptides of the combination caused either partial or delayed tumor regression in xenograft model. In the present application we tested the in vivo tumor regression of the combination at 3 different dose levels of 8, 25 and 50 μg/mouse.

TABLE IX

| | Amount of peptide (in μg) | | | |
|---|---|---|---|---|
| | | | | SEQ ID |
| Dose | SEQ ID NO:1 | SEQ ID NO:2 | SEQ ID NO:3 | NO:4 |
| 8 μg | 3.42 | 1.14 | 1.14 | 2.28 |
| 25 μg | 10.71 | 3.57 | 3.57 | 7.14 |
| 50 μg | 21.42 | 7.14 | 7.14 | 14.28 |

Briefly, human colon adenocarcinoma (PTC) xenografts were grown in Balb/c athymic mice by subcutaneous inoculation of a single cell suspension of PTC cells (15×10⁶ cells/100 μL). The tumor bearing animals were divided into 4 groups of three animals each including one group comprising untreated control animals. Treatment with 3 different doses of the combination, as mentioned above, was initiated when the average tumor volumes were approximately 1.3 cm³. The treatment was given subcutaneously in two equally divided doses for a period of 14 days.

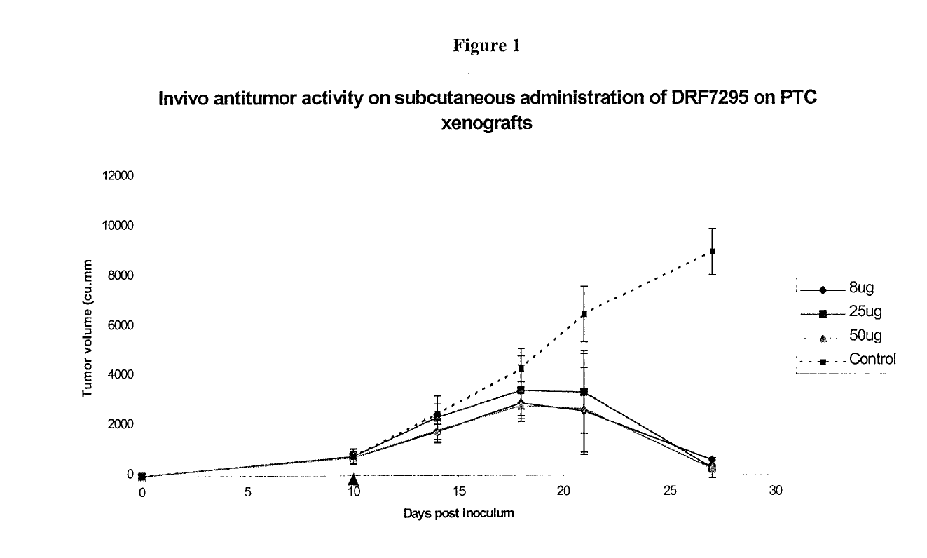

The antitumor activity of the combination was monitored by measuring tumor volumes every fourth day using the formula W*W*L*0.4 (W=smaller diameter, L=larger diameter). The percentage inhibition of tumor growth was calculated using the formula (1−tumor volume-treated/tumor volume-control)*100. FIG. 1 shows the tumor kinetics till day 27 in the treated and untreated animals. The combination showed a significant antitumor activity on PTC xenografts at all three dose levels tested.

The dose levels of 8 to 50 μg/mouse extrapolates to approximately 50-330 μg/Kg. B.Wt in an adult human.

### EXAMPLE 11

### Measurement of MAP Kinase Activity

Confluent PTC cells were harvested using 0.05% trypsin-0.2 mM EDTA and re-plated in RPMI supplemented with 10% FCS, in 6-well tissue culture plates at a density of 50,000 cells/well. After overnight incubation, the medium was changed to RPMI supplemented with 2.5% FCS. Thereafter, the cells were incubated with SEQ ID NO:1+SEQ ID NO:2+SEQ ID NO:3+SEQ ID NO:4 with a final concentration of each peptide being 10⁻⁹ M, for 15 minutes. The cells were washed once with lysis buffer containing 10 mM Tris/150 mM NaCl/2 mM EDTA/2 mM DTT/1 mM orthovanadate/1 mM PMSF/1% Triton-X-100 (pH=7.4). The cellular debris was precipitated at 25000 g & the supernatant retained for MAPK estimation.

The reaction was initiated by the addition of magnesium ATP buffer containing γ-³²P-ATP at a concentration of 200 μCi/ml in presence of the substrate peptide. The mixture was incubated for 30 minutes. The reaction was terminated according to the manufacturer's instructions and the mixture was loaded on treated binding sheets, using the MAP Kinase assay kit (Amersham Pharmacia Biotech, U.S.A). The MAP Kinase activity was calculated from the rate at which phosphate group was transferred to the specific substrate peptide per minute.

The treatment of colon carcinoma cells with SEQ ID NO:1+SEQ ID NO:2+SEQ ID NO:3+SEQ ID NO:4 was found to cause a highly significant downregulation of 81% to 96% of constitutive MAP Kinase activity within 15 minutes of drug treatment.

1. A pharmaceutical composition comprising a therapeutically effective combination of at least two of peptides selected from SEQ ID NO:1 or a pharmaceutically acceptable salt thereof, SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

2. The pharmaceutical composition according to claim 1, wherein the composition comprises SEQ ID NO:1 or a pharmaceutically acceptable salt thereof and SEQ ID NO:2 or a pharmaceutically acceptable salt thereof.

3. The pharmaceutical composition according to claim 1, wherein the composition comprises SEQ ID NO:1 or a pharmaceutically acceptable salt thereof and SEQ ID NO:3 or a pharmaceutically acceptable salt thereof.

4. The pharmaceutical composition according to claim 1, wherein the composition comprises SEQ ID NO:1 or a pharmaceutically acceptable salt thereof and SEQ ID NO:4 or a pharmaceutically acceptable salt thereof.

5. The pharmaceutical composition according to claim 1, wherein the composition comprises SEQ ID NO:2 or a pharmaceutically acceptable salt thereof and SEQ ID NO:3 or a pharmaceutically acceptable salt thereof.

6. The pharmaceutical composition according to claim 1, wherein the composition comprises SEQ ID NO:2 or a pharmaceutically acceptable salt thereof and SEQ ID NO:4 or a pharmaceutically acceptable salt thereof.

7. The pharmaceutical composition according to claim 1, wherein the composition comprises SEQ ID NO:3 or a pharmaceutically acceptable salt thereof and SEQ ID NO:4 or a pharmaceutically acceptable salt thereof.

8. The pharmaceutical composition according to claim 1, wherein the composition comprises SEQ ID NO:1 or a pharmaceutically acceptable salt thereof SEQ ID NO:2 or a pharmaceutically acceptable salt thereof and SEQ ID NO:3 or a pharmaceutically acceptable salt thereof.

9. The pharmaceutical composition according to claim 1, wherein the composition comprises SEQ ID NO:1 or a pharmaceutically acceptable salt thereof SEQ ID NO:3 or a pharmaceutically acceptable salt thereof and SEQ ID NO:4 or a pharmaceutically acceptable salt thereof.

10. The pharmaceutical composition according to claim 1, wherein the composition comprises SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

11. The pharmaceutical composition according to claim 1, wherein the composition comprises SEQ ID NO:1 or a pharmaceutically acceptable salt thereof, SEQ ID NO:2 or a pharmaceutically acceptable salt thereof and SEQ ID NO:4 or a pharmaceutically acceptable salt thereof.

12. The pharmaceutical composition according to claim 1, wherein the composition comprises SEQ ID NO:1 or a pharmaceutically acceptable salt thereof, SEQ ID NO:2 or a pharmaceutically acceptable salt thereof, SEQ ID NO:3 or a pharmaceutically acceptable salt and SEQ ID NO:4 or a pharmaceutical acceptable salt thereof.

13. The pharmaceutical composition as claimed in claim 1, further comprising a pharmaceutically acceptable carrier, diluent or solvent.

14. The pharmaceutical composition as claimed in claim 2, further comprising a pharmaceutically acceptable carrier, diluent or solvent.

15. The pharmaceutical composition as claimed in claim 3, further comprising a pharmaceutically acceptable carrier, diluent or solvent.

16. The pharmaceutical composition as claimed in claim 4, further comprising a pharmaceutically acceptable carrier, diluent or solvent.

17. The pharmaceutical composition as claimed in claim 5, further comprising a pharmaceutically acceptable carrier, diluent or solvent.

18. The pharmaceutical composition as claimed in claim 6, further comprising a pharmaceutically acceptable carrier, diluent or solvent.

19. The pharmaceutical composition as claimed in claim 7, further comprising a pharmaceutically acceptable carrier, diluent or solvent.

20. The pharmaceutical composition as claimed in claim 8, further comprising a pharmaceutically acceptable carrier, diluent or solvent.

21. The pharmaceutical composition as claimed in claim 9, further comprising a pharmaceutically acceptable carrier, diluent or solvent.

22. The pharmaceutical composition as claimed in claim 10, further comprising a pharmaceutically acceptable carrier, diluent or solvent.

23. The pharmaceutical composition as claimed in claim 11, further comprising a pharmaceutically acceptable carrier, diluent or solvent.

24. The pharmaceutical composition as claimed in claim 12 further comprising a pharmaceutically acceptable carrier, diluent or solvent.

25. The pharmaceutical composition as claimed in claim 1, wherein the concentration of each peptide is about 10⁻⁶ M to 10⁻¹⁰ M.

26. The pharmaceutical composition as claimed in claim 2, wherein the concentration of each peptide is about 10⁻⁶ M to 10⁻¹⁰ M.

27. The pharmaceutical composition as claimed in claim 3, wherein the concentration of each peptide is about 10⁻⁶ M to 10⁻¹⁰ M.

28. The pharmaceutical composition as claimed in claim 4, wherein the concentration of each peptide is about 10⁻⁶ M to 10⁻¹⁰ M.

29. The pharmaceutical composition as claimed in claim 5, wherein the concentration of each peptide is about 10⁻⁶ M to 10⁻¹⁰ M.

30. The pharmaceutical composition as claimed in claim 6, wherein the concentration of each peptide is about 10⁻⁶ M to 10⁻¹⁰ M.

31. The pharmaceutical composition as claimed in claim 7, wherein the concentration of each peptide is about 10⁻⁶ M to 10⁻¹⁰ M.

32. The pharmaceutical composition as claimed in claim 8, wherein the concentration of each peptide is about 10⁻⁶ M to 10⁻¹⁰ M.

33. The pharmaceutical composition as claimed in claim 9, wherein the concentration of each peptide is about 10⁻⁶ M to 10⁻¹⁰ M.

34. The pharmaceutical composition as claimed in claim 10, wherein the concentration of each peptide is about 10⁻⁶ M to 10⁻¹⁰ M.

35. The pharmaceutical composition as claimed in claim 11, wherein the concentration of each peptide is about 10⁻⁶ M to 10⁻¹⁰ M.

36. The pharmaceutical composition as claimed in claim 12, wherein the concentration of each peptide is about 10⁻⁶ M to 10⁻¹⁰ M.

37. The pharmaceutical composition as claimed in claim 1, wherein the concentration of each peptide is about 10⁻⁸ M to 10⁻⁹ M.

38. The pharmaceutical composition as claimed in claim 2, wherein the concentration of each peptide is about 10⁻⁸ M to 10⁻⁹ M.

39. The pharmaceutical composition as claimed in claim 3, wherein the concentration of each peptide is about 10⁻⁸ M to 10⁻⁹ M.

40. The pharmaceutical composition as claimed in claim 4, wherein the concentration of each peptide is about 10⁻⁸ M to 10⁻⁹ M.

41. The pharmaceutical composition as claimed in claim 5, wherein the concentration of each peptide is about 10⁻⁸ M to 10⁻⁹ M.

42. The pharmaceutical composition as claimed in claim 6, wherein the concentration of each peptide is about 10⁻⁸ M to 10⁻⁹ M.

43. The pharmaceutical composition as claimed in claim 7, wherein the concentration of each peptide is about 10⁻⁸ M to 10⁻⁹ M.

44. The pharmaceutical composition as claimed in claim 8, wherein the concentration of each peptide is about 10⁻⁸ M to 10⁻⁹ M.

45. The pharmaceutical composition as claimed in claim 9, wherein the concentration of each peptide is about 10⁻⁸ M to 10⁻⁹ M.

46. The pharmaceutical composition as claimed in claim 10, wherein the concentration of each peptide is about 10⁻⁸ M to 10⁻⁹ M.

47. The pharmaceutical composition as claimed in claim 11, wherein the concentration of each peptide is about 10⁻⁸ M to 10⁻⁹ M

48. The pharmaceutical composition as claimed in claim 12, wherein the concentration of each peptide is about 10⁻⁸ M to 10⁻⁹ M.

49. The pharmaceutical composition as claimed in claim 1, wherein the ratio of one peptide is between 1 to 3 times the weight of a second peptide.

50. The pharmaceutical composition as claimed in claim 12, wherein the ratio of one peptide is between 1 to 3 times the weight of a second peptide.

51. The composition as claimed in claim 1, wherein the weight of the peptides in a single dose is between 0.01 to 20.0 mg.

52. The composition as claimed in claim 2, wherein the weight of the peptides in a single dose is between 0.01 to 20.0 mg.

53. The composition as claimed in claim 3, wherein the weight of the peptides in a single dose is between 0.01 to 20.0 mg.

54. The composition as claimed in claim 4, wherein the weight of the peptides in a single dose is between 0.01 to 20.0 mg.

55. The composition as claimed in claim 5, wherein the weight of the peptides in a single dose is between 0.01 to 20.0 mg.

56. The composition as claimed in claim 6, wherein the weight of the peptides in a single dose is between 0.01 to 20.0 mg.

57. The composition as claimed in claim 7, wherein the weight of the peptides in a single dose is between 0.01 to 20.0 mg.

58. The composition as claimed in claim 8, wherein the weight of the peptides in a single dose is between 0.01 to 20.0 mg.

59. The composition as claimed in claim 9, wherein the weight of the peptides in a single dose is between 0.01 to 20.0 mg.

60. The composition as claimed in claim 10, wherein the weight of the peptides in a single dose is between 0.01 to 20.0 mg.

61. The composition as claimed in claim 11, wherein the weight of the peptides in a single dose is between 0.01 to 20.0 mg.

62. The composition as claimed in claim 12, wherein the weight of the peptides in a single dose is between 0.01 to 20.0 mg.

63. A method of killing or inhibiting the multiplication of tumor cells or cancer cells in a human or other animal, the method comprising administering to the human or animal a therapeutically effective combination comprising at least two peptides selected from SEQ ID NO:1 or a pharmaceutically acceptable salt thereof, SEQ ID NO:2 or a pharmaceutically acceptable salt thereof, SEQ ID NO:3 or a pharmaceutically acceptable salt thereof, and SEQ ID NO:4 or a pharmaceutically acceptable salt thereof.

64. A method according to claim 63, wherein the therapeutically effective combination comprises SEQ ID NO:1 or a pharmaceutically acceptable salt thereof and SEQ ID NO:2 or a pharmaceutically acceptable salt thereof.

65. A method according to claim 63, wherein the therapeutically effective combination comprises SEQ ID NO:1 or a pharmaceutically acceptable salt thereof and SEQ ID NO:3 or a pharmaceutically acceptable salt thereof.

66. A method according to claim 63, wherein the therapeutically effective combination comprises SEQ ID NO:1 or a pharmaceutically acceptable salt thereof and SEQ ID NO:4 or a pharmaceutically acceptable salt thereof.

67. A method according to claim 63, wherein the therapeutically effective combination comprises SEQ ID NO:2 or a pharmaceutically acceptable salt thereof and SEQ ID NO:3 or a pharmaceutically acceptable salt thereof.

68. A method according to claim 63, wherein the therapeutically effective combination comprises SEQ ID NO:2 or a pharmaceutically acceptable salt thereof and SEQ ID NO:4 or a pharmaceutically acceptable salt thereof.

69. A method according to claim 63, wherein the therapeutically effective combination comprises SEQ ID NO:3 or a pharmaceutically acceptable salt thereof and SEQ ID NO:4 or a pharmaceutically acceptable salt thereof.

70. A method according to claim 63, wherein the therapeutically effective combination comprises SEQ ID NO:1 or a pharmaceutically acceptable salt thereof, SEQ ID NO:2 or a pharmaceutically acceptable salt thereof and SEQ ID NO:3 or a pharmaceutically acceptable salt thereof.

71. A method according to claim 63, wherein the therapeutically effective combination comprises SEQ ID NO:1 or a pharmaceutically acceptable salt thereof, SEQ ID NO:3 or a pharmaceutically acceptable salt thereof and SEQ ID NO:4 or a pharmaceutically acceptable salt thereof.

72. A method according to claim 63, wherein the therapeutically effective combination comprises SEQ ID NO:1 or a pharmaceutically acceptable salt thereof, SEQ ID NO:2 or a pharmaceutically acceptable salt thereof and SEQ ID NO:4 or a pharmaceutically acceptable salt thereof.

73. A method according to claim 63, wherein the therapeutically effective combination comprises SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

74. The method according to claim 63, wherein the therapeutically effective combination comprises SEQ ID NO:1 or a pharmaceutically acceptable salt thereof, SEQ iD NO:2 or a pharmaceutically acceptable salt thereof, SEQ ID NO:3 or a pharmaceutically acceptable salt thereof and SEQ ID NO:4 or a pharmaceutically acceptable salt thereof.

75. The method according to claim 63, wherein the concentration of each peptide in the combination is about 10⁻⁶ M to 10⁻¹⁰ M.

76. The method according to claim 64, wherein the concentration of each peptide in the combination is about 10⁻⁶ M to 10⁻¹⁰ M.

77. The method according to claim 65, wherein the concentration of each peptide in the combination is about 10⁻⁶ M to 10⁻¹⁰ M.

78. The method according to claim 66, wherein the concentration of each peptide in the combination is about 10⁻⁶ M to 10⁻¹⁰ M.

79. The method according to claim 67, wherein the concentration of each peptide in the combination is about 10⁻⁶ M to 10⁻¹⁰ M.

80. The method according to claim 68, wherein the concentration of each peptide in the combination is about 10⁻⁶ M to 10⁻¹⁰ M.

81. The method according to claim 69, wherein the concentration of each peptide in the combination is about 10⁻⁶ M to 10⁻¹⁰ M.

82. The method according to claim 70, wherein the concentration of each peptide in the combination is about 10⁻⁶ M to 10⁻¹⁰ M.

83. The method according to claim 71, wherein the concentration of each peptide in the combination is about 10⁻⁶ M to 10⁻¹⁰ M.

84. The method according to claim 72, wherein the concentration of each peptide in the combination is about 10⁻⁶ M to 10⁻¹⁰ M.

85. The method according to claim 73, wherein the concentration of each peptide in the combination is about 10⁻⁶ M to 10⁻¹⁰ M.

86. The method according to claim 74, wherein the concentration of each peptide in the combination is about 10⁻⁶ M to 10⁻¹⁰ M.

87. The method as claimed in claim 63, wherein the concentration of each peptide in the combination is in the range of 10⁻⁸ M to 10⁻⁹M.

88. The method as claimed in claim 64, wherein the concentration of each peptide in the combination is in the range of 10⁻⁸ M to 10⁻⁹M.

89. The method as claimed in claim 65, wherein the concentration of each peptide in the combination is in the range of 10⁻⁸ M to 10⁻⁹M.

90. The method as claimed in claim 66, wherein the concentration of each peptide in the combination is in the range of 10⁻⁸ M to 10⁻⁹M.

91. The method as claimed in claim 67, wherein the concentration of each peptide in the combination is in the range of 10⁻⁸ M to 10⁻⁹M.

92. The method as claimed in claim 68, wherein the concentration of each peptide in the combination is in the range of 10⁻⁸ M to 10⁻⁹M.

93. The method as claimed in claim 69, wherein the concentration of each peptide in the combination is in the range of 10⁻⁸ M to 10⁻⁹M.

94. The method as claimed in claim 70, wherein the concentration of each peptide in the combination is in the range of 10⁻⁸ M to 10⁻⁹M.

95. The method as claimed in claim 71, wherein the concentration of each peptide in the combination is in the range of 10⁻⁸ M to 10⁻⁹M.

96. The method as claimed in claim 72, wherein the concentration of each peptide in the combination is in the range of 10⁻⁸ M to 10⁻⁹M.

97. The method as claimed in claim 73, wherein the concentration of each peptide in the combination is in the range of 10⁻⁸ M to 10⁻⁹M.

98. The method as claimed in claim 74, wherein the concentration of each peptide in the combination is in the range of 10⁻⁸ M to 10⁻⁹M.

99. A method for inducing caspase enzyme comprising administering to a human or animal a therapeutically effective combination of peptides comprising at least two peptides selected from SEQ ID NO:1 or a pharmaceutically acceptable salt thereof, SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

100. A method according to claim 99, wherein the therapeutically effective combination comprises SEQ ID NO: 1 or a pharmaceutically acceptable salt thereof and SEQ ID NO: 2 or a pharmaceutically acceptable salt thereof.

101. A method according to claim 99, wherein the therapeutically effective combination comprises SEQ ID NO: 1 or a pharmaceutically acceptable salt thereof and SEQ ID NO: 3 or a pharmaceutically acceptable salt thereof.

102. A method according to claim 99, wherein the therapeutically effective combination comprises SEQ ID NO: 1 or a pharmaceutically acceptable salt thereof and SEQ ID NO: 4 or a pharmaceutically acceptable salt thereof.

103. A method according to claim 99, wherein the therapeutically effective combination comprises SEQ ID NO:2 or a pharmaceutically acceptable salt thereof and SEQ ID NO:3 or a pharmaceutically acceptable salt thereof.

104. A method according to claim 99, wherein the therapeutically effective combination comprises SEQ ID NO:2 or a pharmaceutically acceptable salt thereof and SEQ ID NO:4 or a pharmaceutically acceptable salt thereof.

105. A method according to claim 99, wherein the therapeutically effective combination comprises SEQ ID NO:3 or a pharmaceutically acceptable salt thereof and SEQ ID NO:4 or a pharmaceutically acceptable salt thereof.

106. A method according to claim 99, wherein the therapeutically effective combination comprises SEQ ID NO:1 or a pharmaceutically acceptable salt thereof, SEQ ID NO:2 or a pharmaceutically acceptable salt thereof and SEQ ID NO:3 or a pharmaceutically acceptable salt thereof.

107. A method according to claim 99, wherein the therapeutically effective combination comprises SEQ ID NO: 1 or a pharmaceutically acceptable salt thereof, SEQ ID NO: 3 or a pharmaceutically acceptable salt thereof and SEQ ID NO:4 or a pharmaceutically acceptable salt thereof.

108. A method according to claim 99, wherein the therapeutically effective combination comprises SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

109. A method according to claim 99, wherein the therapeutically effective combination comprises SEQ ID NO: 1 or a pharmaceutically acceptable salt thereof, SEQ ID NO: 2 or a pharmaceutically acceptable salt thereof and SEQ ID NO:4 or a pharmaceutically acceptable salt thereof.

110. A method according to claim 99, wherein the therapeutically effective combination comprises SEQ ID NO: 1 or a pharmaceutically acceptable salt thereof, SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

111. Method according to claim 99, wherein the concentration of each peptide in the combination is about 10⁻⁶ M to 10⁻¹⁰ M.

112. A method according to claim 100, wherein the concentration of each peptide in the combination is about 10⁻⁶ M to 10⁻¹⁰ M.

113. A method according to claim 101, wherein the concentration of each peptide in the combination is about 10⁻⁶ M to 10⁻¹⁰ M.

114. A method according to claim 102, wherein the concentration of each peptide in the combination is about 10⁻⁶ M to 10⁻¹⁰ M.

115. A method according to claim 103, wherein the concentration of each peptide in the combination is about 10⁻⁶ M to 10⁻¹⁰ M.

116. A method according to claim 104, wherein the concentration of each peptide in the combination is about 10⁻⁶ M to 10⁻¹⁰ M.

117. A method according to claim 105, wherein the concentration of each peptide in the combination is about 10⁻⁶ M to 10⁻¹⁰ M.

118. A method according to claim 106, wherein the concentration of each peptide in the combination is about 10⁻⁶ M to 10⁻¹⁰ M.

119. A method according to claim 107, wherein the concentration of each peptide in the combination is about 10⁻⁶ M to 10⁻¹⁰ M.

120. A method according to claim 108, wherein the concentration of each peptide in the combination is about 10⁻⁶ M to 10⁻¹⁰ M.

121. A method according to claim 109, wherein the concentration of each peptide in the combination is about 10⁻⁶ M to 10⁻¹⁰ M.

122. A method according to claim 110, wherein the concentration of each peptide in the combination is about 10⁻⁶ M to 10⁻¹⁰ M.

123. The method as claimed in claim 99, wherein the concentration of each peptide in the combination is in the range of 10⁻⁸ M to 10⁻⁹M.

124. The method as claimed in claim 100, wherein the concentration of each peptide in the combination is in the range of 10⁻⁸ M to 10⁻⁹M.

125. The method as claimed in claim 101, wherein the concentration of each peptide in the combination is in the range of 10⁻⁸ M to 10⁻⁹M.

126. The method as claimed in claim 102, wherein the concentration of each peptide in the combination is in the range of 10⁻⁸ M to 10⁻⁹M.

127. The method as claimed in claim 103, wherein the concentration of each peptide in the combination is in the range of 10⁻⁸ M to 10⁻⁹M.

128. The method as claimed in claim 104, wherein the concentration of each peptide in the combination is in the range of 10⁻⁸ M to 10⁻⁹M.

129. The method as claimed in claim 105, wherein the concentration of each peptide in the combination is in the range of 10⁻⁸ M to 10⁻⁹M.

130. The method as claimed in claim 106, wherein the concentration of each peptide in the combination is in the range of 10⁻⁸ M to 10⁻⁹M.

131. The method as claimed in claim 107, wherein the concentration of each peptide in the combination is in the range of 10⁻⁸ M to 10⁻⁹M.

132. The method as claimed in claim 108, wherein the concentration of each peptide in the combination is in the range of 10⁻⁸ M to 10⁻⁹M.

133. The method as claimed in claim 109, wherein the concentration of each peptide in the combination is in the range of 10⁻⁸ M to 10⁻⁹M.

134. The method as claimed in claim 110, wherein the concentration of each peptide in the combination is in the range of 10⁻⁸ M to 10⁻⁹M.

135. A method for downregulating intracellular levels of cAMP comprising administering to a human or animal a therapeutically effective combination of peptides comprising at least two peptides selected from SEQ ID NO:1 or a pharmaceutically acceptable salt thereof, SEQ ID NO: 2 or a pharmaceutically acceptable salt thereof, SEQ ID NO:3 or a pharmaceutically acceptable salt thereof and SEQ ID NO:4 or a pharmaceutically acceptable salt thereof.

136. A method according to claim 135, wherein the therapeutically effective combination comprises SEQ ID NO: 1 or a pharmaceutically acceptable salt thereof and SEQ ID NO: 2 or a pharmaceutically acceptable salt thereof.

137. A method according to claim 135, wherein the therapeutically effective combination comprises SEQ ID NO: 2 or a pharmaceutically acceptable salt thereof and SEQ ID NO: 3 or a pharmaceutically acceptable salt thereof.

138. A method according to claim 135, wherein the therapeutically effective combination comprises SEQ ID NO: 1 or a pharmaceutically acceptable salt thereof and SEQ ID NO: 4 or a pharmaceutically acceptable salt thereof.

139. A method according to claim 135, wherein the therapeutically effective combination comprises SEQ ID NO:2 or a pharmaceutically acceptable salt thereof and SEQ ID NO:3 or a pharmaceutically acceptable salt thereof.

140. A method according to claim 135, wherein the therapeutically effective combination comprises SEQ ID NO:2 or a pharmaceutically acceptable salt thereof and SEQ ID NO:4 or a pharmaceutically acceptable salt thereof.

141. A method according to claim 135, wherein the therapeutically effective combination comprises SEQ ID NO:3 or a pharmaceutically acceptable salt thereof and SEQ ID NO:4 or a pharmaceutically acceptable salt thereof.

142. A method according to claim 135, wherein the therapeutically effective combination comprises SEQ ID NO: 1 or a pharmaceutically acceptable salt thereof SEQ ID NO:2 or a pharmaceutically acceptable salt thereof and SEQ ID NO:3 or a pharmaceutically acceptable salt thereof.

143. A method according to claim 135, wherein the therapeutically effective combination comprises SEQ ID NO: 1 or a pharmaceutically acceptable salt thereof SEQ ID NO: 3 or a pharmaceutically acceptable salt thereof and SEQ ID NO:4 or a pharmaceutically acceptable salt thereof.

144. A method according to claim 135, wherein the therapeutically effective combination comprises SEQ ID NO: 2 or a pharmaceutically acceptable salt thereof, SEQ ID NO: 3 or a pharmaceutically acceptable salt thereof and SEQ ID NO:5 or a pharmaceutically acceptable salt thereof.

145. A method according to claim 135, wherein the therapeutically effective combination comprises SEQ ID NO: 1 or a pharmaceutically acceptable salt thereof, SEQ ID NO: 2 or a pharmaceutically acceptable salt thereof and SEQ ID NO: 4 or a pharmaceutically acceptable salt thereof.

146. A method according to claim 135, wherein the therapeutically effective combination comprises SEQ ID NO: 1 or a pharmaceutically acceptable salt thereof, SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

147. The method according to claim 135, wherein the concentration of each peptide in the combination is about 10⁻⁶ M to 10⁻¹⁰ M.

148. A method according to claim 136, wherein the concentration of each peptide in the combination is about 10⁻⁶ M to 10⁻¹⁰ M.

149. A method according to claim 137, wherein the concentration of each peptide in the combination is about 10⁻⁶ M to 10⁻¹⁰ M.

150. A method according to claim 138, wherein the concentration of each peptide in the combination is about 10⁻⁶ M to 10⁻¹⁰ M.

151. A method according to claim 139, wherein the concentration of each peptide in the combination is about 10⁻⁶ M to 10⁻¹⁰ M.

152. A method according to claim 140, wherein the concentration of each peptide in the combination is about 10⁻⁶ M to 10⁻¹⁰ M.

153. A method according to claim 141, wherein the concentration of each peptide in the combination is about 10⁻⁶ M to 10⁻¹⁰ M.

154. A method according to claim 142, wherein the concentration of each peptide in the combination is about 10⁻⁶ M to 10⁻¹⁰ M.

155. A method according to claim 143, wherein the concentration of each peptide in the combination is about 10⁻⁶ M to 10⁻¹⁰ M.

156. A method according to claim 144, wherein the concentration of each peptide in the combination is about 10⁻⁶ M to 10⁻¹⁰ M.

157. A method according to claim 145, wherein the concentration of each peptide in the combination is about 10⁻⁶ M to 10⁻¹⁰ M.

158. A method according to claim 146, wherein the concentration of each peptide in the combination is about 10⁻⁶ M to 10⁻¹⁰ M.

159. The method as claimed in claim 135, wherein the concentration of each peptide in the combination is in the range of 10⁻⁸ M to 10⁻⁹M.

160. The method as claimed in claim 136, wherein the concentration of each peptide in the combination is in the range of 10⁻⁸ M to 10⁻⁹M.

161. The method as claimed in claim 137, wherein the concentration of each peptide in the combination is in the range of 10⁻⁸ M to 10⁻⁹M.

162. The method as claimed in claim 138, wherein the concentration of each peptide in the combination is in the range of 10⁻⁸ M to 10⁻⁹M.

163. The method as claimed in claim 139, wherein the concentration of each peptide in the combination is in the range of 10⁻⁸ M to 10⁻⁹M.

164. The method as claimed in claim 140, wherein the concentration of each peptide in the combination is in the range of 10⁻⁸ M to 10⁻⁹M.

165. The method as claimed in claim 141, wherein the concentration of each peptide in the combination is in the range of 10⁻⁸ M to 10⁻⁹M.

166. The method as claimed in claim 142, wherein the concentration of each peptide in the combination is in the range of 10⁻⁸ M to 10⁻⁹M.

167. The method as claimed in claim 143, wherein the concentration of each peptide in the combination is in the range of 10⁻⁸ M to 10⁻⁹M.

168. The method as claimed in claim 144, wherein the concentration of each peptide in the combination is in the range of 10⁻⁸ M to 10⁻⁹M.

169. The method as claimed in claim 145, wherein the concentration of each peptide in the combination is in the range of 10⁻⁸ M to 10⁻⁹M.

170. The method as claimed in claim 146, wherein the concentration of each peptide in the combination is in the range of 10⁻⁸ M to 10⁻⁹M.

171. A method for inhibiting secretion of vascular endothelial growth factor comprising administering to a human or animal a therapeutically effective combination of peptides comprising at least two peptides selected from SEQ ID NO:1 or a pharmaceutically acceptable salt thereof SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

172. The method according to claim 171, wherein the therapeutically effective combination comprises SEQ ID NO: 1 or a pharmaceutically acceptable salt thereof and SEQ ID NO: 2 or a pharmaceutically acceptable salt thereof.

173. The method according to claim 171, wherein the therapeutically effective combination comprises SEQ ID NO: 1 or a pharmaceutically acceptable salt thereof and SEQ ID NO: 3 or a pharmaceutically acceptable salt thereof.

174. The method according to claim 171, wherein the therapeutically effective combination comprises SEQ ID NO: 1 or a pharmaceutically acceptable salt thereof and SEQ ID NO: 4 or a pharmaceutically acceptable salt thereof.

175. The method according to claim 171, wherein the therapeutically effective combination comprises SEQ ID NO:2 or a pharmaceutically acceptable salt thereof and SEQ ID NO:3 or a pharmaceutically acceptable salt thereof.

176. The method according to claim 171, wherein the therapeutically effective combination comprises SEQ ID NO:2 or a pharmaceutically acceptable salt thereof and SEQ ID NO:4 or a pharmaceutically acceptable salt thereof.

177. The method according to claim 171, wherein the therapeutically effective combination comprises SEQ ID NO:3 or a pharmaceutically acceptable salt thereof and SEQ ID NO:4 or a pharmaceutically acceptable salt thereof.

178. The method according to claim 171, wherein the therapeutically effective combination comprises SEQ ID NO: 1 or a pharmaceutically acceptable salt thereof SEQ ID NO:2 or a pharmaceutically acceptable salt thereof and SEQ ID NO:3 or a pharmaceutically acceptable salt thereof.

179. The method according to claim 171, wherein the therapeutically effective combination comprises SEQ ID NO: 1 or a pharmaceutically acceptable salt thereof, SEQ ID NO: 3 or a pharmaceutically acceptable salt thereof and SEQ ID NO:4 or a pharmaceutically acceptable salt thereof.

180. The method according to claim 171, wherein the therapeutically effective combination comprises SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

181. The method according to claim 171, wherein the therapeutically effective combination comprises SEQ ID NO: 1 or a pharmaceutically acceptable salt thereof, SEQ ID NO: 2 or a pharmaceutically acceptable salt thereof and SEQ ID NO:4 or a pharmaceutically acceptable salt thereof.

182. The method according to claim 171, wherein the therapeutically effective combination comprises SEQ ID NO: 1 or a pharmaceutically acceptable salt thereof, SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

183. The method according to claim 171, wherein the concentration of each peptide in the combination is about 10⁻⁶ M to 10⁻¹⁰ M.

184. The method according to claim 172, wherein the concentration of each peptide in the combination is about 10⁻⁶ M to 10⁻¹⁰ M.

185. The method according to claim 173, wherein the concentration of each peptide in the combination is about 10⁻⁶ M to 10⁻¹⁰ M.

186. The method according to claim 174, wherein the concentration of each peptide in the combination is about 10⁻⁶ M to 10⁻¹⁰ M.

187. The method according to claim 175, wherein the concentration of each peptide in the combination is about 10⁻⁶ M to 10⁻¹⁰ M.

188. The method according to claim 176, wherein the concentration of each peptide in the combination is about 10⁻⁶ M to 10⁻¹⁰ M.

189. The method according to claim 177, wherein the concentration of each peptide in the combination is about 10⁻⁶ M to 10⁻¹⁰ M.

190. The method according to claim 178, wherein the concentration of each peptide in the combination is about 10⁻⁶ M to 10⁻¹⁰ M.

191. The method according to claim 179, wherein the concentration of each peptide in the combination is about 10⁻⁶ M to 10⁻¹⁰ M.

192. The method according to claim 180, wherein the concentration of each peptide in the combination is about 10⁻⁶ M to 10⁻¹⁰ M.

193. The method according to claim 181, wherein the concentration of each peptide in the combination is about 10⁻⁶ M to 10⁻¹⁰ M.

194. The method according to claim 182, wherein the concentration of each peptide in the combination is about 10⁻⁶ M to 10⁻¹⁰ M.

195. The method as claimed in claim 171, wherein the concentration of each peptide in the combination is in the range of 10⁻⁸ M to 10⁻⁹M.

196. The method as claimed in claim 172, wherein the concentration of each peptide in the combination is in the range of 10 ⁸ M to 10⁻⁹M.

197. The method as claimed in claim 173, wherein the concentration of each peptide in the combination is in the range of 10⁻⁸ M to 10⁻⁹M.

198. The method as claimed in claim 174, wherein the concentration of each peptide in the combination is in the range of 10⁻⁸ M to 10⁻⁹M.

199. The method as claimed in claim 175, wherein the concentration of each peptide in the combination is in the range of 10⁻⁸ M to 10⁻⁹M.

200. The method as claimed in claim 176, wherein the concentration of each peptide in the combination is in the range of 10⁻⁸ M to 10⁻⁹M.

201. The method as claimed in claim 177, wherein the concentration of each peptide in the combination is in the range of 10⁻⁸ M to 10⁻⁹M.

202. The method as claimed in claim 178, wherein the concentration of each peptide in the combination is in the range of 10⁻⁸ M to 10⁻⁹M.

203. The method as claimed in claim 179, wherein the concentration of each peptide in the combination is in the range of 10⁻⁸ M to 10⁻⁹M.

204. The method as claimed in claim 180, wherein the concentration of each peptide in the combination is in the range of 10⁻⁸ M to 10⁻⁹M.

205. The method as claimed in claim 181, wherein the concentration of each peptide in the combination is in the range of 10⁻⁸ M to 10⁻⁹M.

206. The method as claimed in claim 182, wherein the concentration of each peptide in the combination is in the range of 10⁻⁸ M to 10⁻⁹M.

207. A method for down regulating intracellular levels of mitogen activated protein kinase (MAPK) administering to the human or animal a therapeutically effective combination comprising at least two peptides selected from SEQ ID NO:1 or a pharmaceutically acceptable salt thereof, SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

208. The method according to claim 207, wherein the therapeutically effective combination comprises SEQ ID NO: 1 or a pharmaceutically acceptable salt thereof and SEQ ID NO: 2 or a pharmaceutically acceptable salt thereof.

209. The method according to claim 207, wherein the therapeutically effective combination comprises SEQ ID NO: 2 or a pharmaceutically acceptable salt thereof and SEQ ID NO: 3 or a pharmaceutically acceptable salt thereof.

210. The method according to claim 207, wherein the therapeutically effective combination comprises SEQ ID NO: 1 or a pharmaceutically acceptable salt thereof and SEQ ID NO: 4 or a pharmaceutically acceptable salt thereof.

211. The method according to claim 207, wherein the therapeutically effective combination comprises SEQ ID NO:2 or a pharmaceutically acceptable salt thereof and SEQ ID NO:3 or a pharmaceutically acceptable salt thereof.

212. The method according to claim 207, wherein the therapeutically effective combination comprises SEQ ID NO:2 or a pharmaceutically acceptable salt thereof and SEQ ID NO:4 or a pharmaceutically acceptable salt thereof.

213. The method according to claim 207, wherein the therapeutically effective combination comprises SEQ ID NO:3 or a pharmaceutically acceptable salt thereof and SEQ ID NO:4 or a pharmaceutically acceptable salt thereof.

214. The method according to claim 207, wherein the therapeutically effective combination comprises SEQ ID NO: 1 or a pharmaceutically acceptable salt thereof, SEQ ID NO:2 or a pharmaceutically acceptable salt thereof and SEQ ID NO:3 or a pharmaceutically acceptable salt thereof.

215. The method according to claim 207, wherein the therapeutically effective combination comprises SEQ ID NO: 1 or a pharmaceutically acceptable salt thereof, SEQ ID NO: 3 or a pharmaceutically acceptable salt thereof and SEQ ID NO:4 or a pharmaceutically acceptable salt thereof.

216. The method according to claim 207, wherein the therapeutically effective combination comprises SEQ ID NO: 1 or a pharmaceutically acceptable salt thereof, SEQ ID NO: 2 or a pharmaceutically acceptable salt thereof and SEQ ID NO:4 or a pharmaceutically acceptable salt thereof.

217. The method according to claim 207, wherein the therapeutically effective combination comprises SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

218. The method according to claim 207, wherein the therapeutically effective combination comprises SEQ ID NO: 1 or a pharmaceutically acceptable salt thereof, SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

219. The method according to claim 207, wherein the concentration of each peptide in the combination is about 10⁻⁶ M to 10⁻¹⁰ M.

220. The method according to claim 208, wherein the concentration of each peptide in the combination is about 10⁻⁶ M to 10⁻¹⁰ M.

221. The method according to claim 209, wherein the concentration of each peptide in the combination is about 10⁻⁶ M to 10⁻¹⁰ M.

222. The method according to claim 210, wherein the concentration of each peptide in the combination is about 10⁻⁶ M to 10⁻¹⁰ M.

223. The method according to claim 211, wherein the concentration of each peptide in the combination is about 10⁻⁶ M to 10⁻¹⁰ M.

224. The method according to claim 212, wherein the concentration of each peptide in the combination is about 10⁻⁶ M to 10⁻¹⁰ M.

225. The method according to claim 213, wherein the concentration of each peptide in the combination is about 10⁻⁶ M to 10⁻¹⁰ M.

226. The method according to claim 214, wherein the concentration of each peptide in the combination is about 10⁻⁶ M to 10⁻¹⁰ M.

227. The method according to claim 215, wherein the concentration of each peptide in the combination is about 10⁻⁶ M to 10⁻¹⁰ M.

228. The method according to claim 216, wherein the concentration of each peptide in the combination is about 10⁻⁶ M to 10⁻¹⁰ M.

229. The method according to claim 217, wherein the concentration of each peptide in the combination is about 10⁻⁶ M to 10⁻¹⁰ M.

230. The method according to claim 218, wherein the concentration of each peptide in the combination is about 10⁻⁶ M to 10⁻¹⁰ M.

231. The method as claimed in claim 207, wherein the concentration of each peptide in the combination is in the range of 10⁻⁸ M to 10⁻⁹M.

232. The method as claimed in claim 208, wherein the concentration of each peptide in the combination is in the range of 10⁻⁸ M to 10⁻⁹M.

233. The method as claimed in claim 209, wherein the concentration of each peptide in the combination is in the range of 10⁻⁸ M to 10⁻⁹M.

234. The method as claimed in claim 210, wherein the concentration of each peptide in the combination is in the range of 10⁻⁸ M to 10⁻⁹M.

235. The method as claimed in claim 211, wherein the concentration of each peptide in the combination is in the range of 10⁻⁸ M to 10⁻⁹M.

236. The method as claimed in claim 212, wherein the concentration of each peptide in the combination is in the range of 10⁻⁸ M to 10 ⁹M.

237. The method as claimed in claim 213, wherein the concentration of each peptide in the combination is in the range of 10⁻⁸ M to 10⁻⁹M.

238. The method as claimed in claim 214, wherein the concentration of each peptide in the combination is in the range of 10⁻⁸ M to 10⁻⁹M.

239. The method as claimed in claim 215, wherein the concentration of each peptide in the combination is in the range of 10⁻⁸ M to 10⁻⁹M.

240. The method as claimed in claim 216, wherein the concentration of each peptide in the combination is in the range of 10⁻⁸ M to 10⁻⁹M.

241. The method as claimed in claim 217, wherein the concentration of each peptide in the combination is in the range of 10⁻⁸ M to 10⁻⁹M.

242. The method as claimed in claim 218, wherein the concentration of each peptide in the combination is in the range of 10⁻⁸ M to 10⁻⁹M.

243. A method for upregulating intracellular levels tyrosine phosphatase comprising administering to a human or animal a therapeutically effective combination compiling at least two peptides selected from SEQ ID NO:1 or a pharmaceutically acceptable salt thereof, SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

244. The method according to claim 243, wherein the therapeutically effective combination comprises SEQ ID NO: 1 or a pharmaceutically acceptable salt thereof and SEQ ID NO: 2 or a pharmaceutically acceptable salt thereof.

245. The method according to claim 243, wherein the therapeutically effective combination comprises SEQ ID NO: 2 or a pharmaceutically acceptable salt thereof and SEQ ID NO: 3 or a pharmaceutically acceptable salt thereof.

246. The method according to claim 243, wherein the therapeutically effective combination comprises SEQ ID NO: 1 or a pharmaceutically acceptable salt thereof and SEQ ID NO: 4 or a pharmaceutically acceptable salt thereof.

247. The method according to claim 243, wherein the therapeutically effective combination comprises SEQ ID NO:2 or a pharmaceutically acceptable salt thereof and SEQ ID NO:3 or a pharmaceutically acceptable salt thereof.

248. The method according to claim 243, wherein the therapeutically effective combination comprises SEQ ID NO:2 or a pharmaceutically acceptable salt thereof and SEQ ID NO:4 or a pharmaceutically acceptable salt thereof.

249. The method according to claim 243, wherein the therapeutically effective combination comprises SEQ ID NO:3 or a pharmaceutically acceptable salt thereof and SEQ ID NO:4 or a pharmaceutically acceptable salt thereof.

250. The method according to claim 243, wherein the therapeutically effective combination comprises SEQ ID NO: 1 or a pharmaceutically acceptable salt thereof, SEQ ID NO:2 or a pharmaceutically acceptable salt thereof and SEQ ID NO:3 or a pharmaceutically acceptable salt thereof.

251. The method according to claim 243, wherein the therapeutically effective combination comprises SEQ ID NO: 1 or a pharmaceutically acceptable salt thereof, SEQ ID NO: 3 or a pharmaceutically acceptable salt thereof and SEQ ID NO:4 or a pharmaceutically acceptable salt thereof.

252. The method according to claim 243, wherein the therapeutically effective combination comprises SEQ ID NO: 1 or a pharmaceutically acceptable salt thereof, SEQ ID NO: 2 or a pharmaceutically acceptable salt thereof and SEQ ID NO:4 or a pharmaceutically acceptable salt thereof.

253. The method according to claim 243, wherein the therapeutically effective combination comprises SEQ ID NO: 2 or a pharmaceutically acceptable salt thereof, SEQ ID NO: 3 or a pharmaceutically acceptable salt thereof and SEQ ID NO:5 or a pharmaceutically acceptable salt thereof.

254. The method according to claim 243, wherein the therapeutically effective combination comprises SEQ ID NO: 1 or a pharmaceutically acceptable salt thereof, SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

255. The method according to claim 243, wherein the concentration of each peptide in the combination is about 10⁻⁶ M to 10⁻¹⁰ M.

256. The method according to claim 244, wherein the concentration of each peptide in the combination is about 10⁻⁶ M to 10⁻¹⁰ M.

257. The method according to claim 245, wherein the concentration of each peptide in the combination is about 10⁻⁶ M to 10⁻¹⁰ M.

258. The method according to claim 246, wherein the concentration of each peptide in the combination is about 10⁻⁶ M to 10⁻¹⁰ M.

259. The method according to claim 247, wherein the concentration of each peptide in the combination is about 10⁻⁶ M to 10⁻¹⁰ M.

260. The method according to claim 248, wherein the concentration of each peptide in the combination is about 10⁻⁶ M to 10⁻¹⁰ M.

261. The method according to claim 249, wherein the concentration of each peptide in the combination is about 10⁻⁶ M to 10⁻¹⁰ M.

262. The method according to claim 250, wherein the concentration of each peptide in the combination is about 10⁻⁶ M to 10⁻¹⁰ M.

263. The method according to claim 251, wherein the concentration of each peptide in the combination is about 10⁻⁶ M to 10⁻¹⁰ M.

264. The method according to claim 252, wherein the concentration of each peptide in the combination is about 10⁻⁶ M to 10⁻¹⁰ M.

265. The method according to claim 253, wherein the concentration of each peptide in the combination is about 10⁻⁶ M to 10⁻¹⁰ M.

266. The method according to claim 254, wherein the concentration of each peptide in the combination is about 10⁻⁶ M to 10⁻¹⁰ M.

267. The method as claimed in claim 243, wherein the concentration of each peptide in the combination is in the range of 10⁻⁸ M to 10⁻⁹M.

268. The method as claimed in claim 244, wherein the concentration of each peptide in the combination is in the range of 10⁻⁸ M to 10⁻⁹M.

269. The method as claimed in claim 245, wherein the concentration of each peptide in the combination is in the range of 10⁻⁸ M to 10⁻⁹M.

270. The method as claimed in claim 246, wherein the concentration of each peptide in the combination is in the range of 10⁻⁸ M to 10⁻⁹M.

271. The method as claimed in claim 247, wherein the concentration of each peptide in the combination is in the range of 10⁻⁸ M to 10⁻⁹M.

272. The method as claimed in claim 248, wherein the concentration of each peptide in the combination is in the range of 10⁻⁸ M to 10⁻⁹M.

273. The method as claimed in claim 249, wherein the concentration of each peptide in the combination is in the range of 10⁻⁸ M to 10⁻⁹M.

274. The method as claimed in claim 250, wherein the concentration of each peptide in the combination is in the range of 10⁻⁸ M to 10⁻⁹M.

275. The method as claimed in claim 251, wherein the concentration of each peptide in the combination is in the range of 10⁻⁸ M to 10⁻⁹M.

276. The method as claimed in claim 252, wherein the concentration of each peptide in the combination is in the range of 10⁻⁸ M to 10⁻⁹M.

277. The method as claimed in claim 253, wherein the concentration of each peptide in the combination is in the range of 10⁻⁸ M to 10⁻⁹M.

278. The method as claimed in claim 254, wherein the concentration of each peptide in the combination is in the range of 10⁻⁸ M to 10⁻⁹M.

279. A method for down regulating epidermal growth factor dependent proliferation comprising administering to the human or animal a therapeutically effective combination comprising at least two peptides selected from SEQ ID NO:1 or a pharmaceutically acceptable salt thereof, SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

280. The method according to claim 279, wherein the therapeutically effective combination comprises SEQ ID NO: 1 or a pharmaceutically acceptable salt thereof and SEQ ID NO: 2 or a pharmaceutically acceptable salt thereof.

281. The method according to claim 279, wherein the therapeutically effective combination comprises SEQ ID NO: 1 or a pharmaceutically acceptable salt thereof and SEQ ID NO: 3 or a pharmaceutically acceptable salt thereof.

282. The method according to claim 279, wherein the therapeutically effective combination comprises SEQ ID NO: 1 or a pharmaceutically acceptable salt thereof and SEQ ID NO: 4 or a pharmaceutically acceptable salt thereof.

283. The method according to claim 279, wherein the therapeutically effective combination comprises SEQ ID NO:2 or a pharmaceutically acceptable salt thereof and SEQ ID NO:3 or a pharmaceutically acceptable salt thereof.

284. The method according to claim 279, wherein the therapeutically effective combination comprises SEQ ID NO:2 or a pharmaceutically acceptable salt thereof and SEQ ID NO:4 or a pharmaceutically acceptable salt thereof.

285. The method according to claim 279, wherein the therapeutically effective combination comprises SEQ ID NO:3 or a pharmaceutically acceptable salt thereof and SEQ ID NO:4 or a pharmaceutically acceptable salt thereof.

286. The method according to claim 279, wherein the therapeutically effective combination comprises SEQ ID NO: 1 or a pharmaceutically acceptable salt thereof, SEQ ID NO:2 or a pharmaceutically acceptable salt thereof and SEQ ID NO:3 or a pharmaceutically acceptable salt thereof.

287. The method according to claim 279, wherein the therapeutically effective combination comprises SEQ ID NO: 1 or a pharmaceutically acceptable salt thereof, SEQ ID NO: 3 or a pharmaceutically acceptable salt thereof and SEQ ID NO:4 or a pharmaceutically acceptable salt thereof.

288. The method according to claim 279, wherein the therapeutically effective combination comprises SEQ ID NO: 2 or a pharmaceutically acceptable salt thereof, SEQ ID NO: 3 or a pharmaceutically acceptable salt thereof and SEQ ID NO:5 or a pharmaceutically acceptable salt thereof.

289. The method according to claim 279, wherein the therapeutically effective combination comprises SEQ ID NO: 1 or a pharmaceutically acceptable salt thereof, SEQ ID NO: 2 or a pharmaceutically acceptable salt thereof and SEQ ID NO:4 or a pharmaceutically acceptable salt thereof.

290. The method according to claim 279, wherein the therapeutically effective combination comprises SEQ ID NO: 1 or a pharmaceutically acceptable salt thereof, SEQ ID NO: 2 or a pharmaceutically acceptable salt thereof, SEQ ID NO: 3 or a pharmaceutically acceptable salt thereof and SEQ ID NO:4 or a pharmaceutically acceptable salt thereof.

291. The method according to claim 279, wherein the concentration of each peptide in the combination is about 10⁻⁶ M to 10⁻¹⁰ M.

292. A method according to claim 280, wherein the concentration of each peptide in the combination is about 10⁻⁶ M to 10⁻¹⁰ M.

293. A method according to claim 281, wherein the concentration of each peptide in the combination is about 10⁻⁶ M to 10⁻¹⁰ M.

294. A method according to claim 282, wherein the concentration of each peptide in the combination is about 10⁻⁶ M to 10⁻¹⁰ M.

295. A method according to claim 283, wherein the concentration of each peptide in the combination is about 10⁻⁶ M to 10⁻¹⁰ M.

296. A method according to claim 284, wherein the concentration of each peptide in the combination is about 10⁻⁶ M to 10⁻¹⁰ M.

297. A method according to claim 285, wherein the concentration of each peptide in the combination is about 10⁻⁶ M to 10⁻¹⁰ M.

298. A method according to claim 286, wherein the concentration of each peptide in the combination is about 10⁻⁶ M to 10⁻¹⁰ M.

299. A method according to claim 287, wherein the concentration of each peptide in the combination is about 10⁻⁶ M to 10⁻¹⁰ M.

300. A method according to claim 288, wherein the concentration of each peptide in the combination is about 10⁻⁶ M to 10⁻¹⁰ M.

301. A method according to claim 289, wherein the concentration of each peptide in the combination is about 10⁻⁶ M to 10⁻¹⁰ M.

302. A method according to claim 290, wherein the concentration of each peptide in the combination is about 10⁻⁶ M to 10⁻¹⁰ M.

303. The method as claimed in claim 279, wherein the concentration of each peptide in the combination is in the range of 10⁻⁸ M to 10⁻⁹M.

304. The method as claimed in claim 280, wherein the concentration of each peptide in the combination is in the range of 10⁻⁸ M to 10⁻⁹M.

305. The method as claimed in claim 281, wherein the concentration of each peptide in the combination is in the range of 10⁻⁸ M to 10⁻⁹M.

306. The method as claimed in claim 282, wherein the concentration of each peptide in the combination is in the range of 10⁻⁸ M to 10⁻⁹M.

307. The method as claimed in claim 283, wherein the concentration of each peptide in the combination is in the range of 10⁻⁸ M to 10⁻⁹M.

308. The method as claimed in claim 284, wherein the concentration of each peptide in the combination is in the range of 10₋₈ M to 10⁻⁹M.

309. The method as claimed in claim 285, wherein the concentration of each peptide in the combination is in the range of 10⁻⁸ M to 10⁻⁹M.

310. The method as claimed in claim 286, wherein the concentration of each peptide in the combination is in the range of 10⁻⁸ M to 10⁻⁹M.

311. The method as claimed in claim 287, wherein the concentration of each peptide in the combination is in the range of 10⁻⁸ M to 10⁻⁹M.

312. The method as claimed in claim 288, wherein the concentration of each peptide in the combination is in the range of 10⁻⁸ M to 10⁻⁹M.

313. The method as claimed in claim 289, wherein the concentration of each peptide in the combination is in the range of 10⁻⁸ M to 10⁻⁹M.

314. The method as claimed in claim 290, wherein the concentration of each peptide in the combination is in the range of 10⁻⁸ M to 10⁻⁹M.

315. A pharmaceutical composition according to claim 1, wherein a single dose is 0.05-500 ug of peptides/Kg of body weight.

316. A pharmaceutical composition according to claim 2, wherein a single dose is 0.05-500 ug of peptides/Kg of body weight.

317. A pharmaceutical composition according to claim 3, wherein a single dose is 0.05-500 ug of peptides/Kg of body weight.

318. A pharmaceutical composition according to claim 4, wherein a single dose is 0.05-500 ug of peptides/Kg of body weight.

319. A pharmaceutical composition according to claim 5, wherein a single dose is 0.05-500 ug of peptides/Kg of body weight.

320. A pharmaceutical composition according to claim 6, wherein a single dose is 0.05-500 ug of peptides/Kg of body weight.

321. A pharmaceutical composition according to claim 7, wherein a single dose is 0.05-500 ug of peptides/Kg of body weight.

322. A pharmaceutical composition according to claim 8, wherein a single dose is 0.05-500 ug of peptides/Kg of body weight.

323. A pharmaceutical composition according to claim 9, wherein a single dose is 0.05-500 ug of peptides/Kg of body weight.

324. A pharmaceutical composition according to claim 10, wherein a single dose is 0.05-500 ug of peptides/Kg of body weight.

325. A pharmaceutical composition according to claim 11, wherein a single dose is 0.05-500 ug of peptides/Kg of body weight.

326. A pharmaceutical composition according to claim 12, wherein a single dose is 0.05-500 ug of peptides/Kg of body weight.

327. A method for treating cancer comprising administering to a human or animal in need thereof comprising administering to the human or animal a therapeutically effective combination of at least two peptides selected from SEQ ID NO:1 or a pharmaceutically acceptable salt thereof, SEQ ID NO:2 or a pharmaceutically acceptable salt thereof, SEQ ID NO:3 or a pharmaceutically acceptable salt thereof and SEQ ID NO:4 or a pharmaceutically acceptable salt thereof.

328. The method according to claim 327, wherein the cancer is breast, ovarian, colon, lung, pancreatic, prostate, stomach or oral, or skin fibroblasts.
